(19) United States
(12) Patent Application Publication
(10) Pub. No.: US 2009/0326622 A1
(43) Pub. Date: Dec. 31, 2009

(54) Customizable therapeutic article for applying heat to the body

(76) Inventors: Kroy D. Johnson, Neenah, WI (US); Frank G. Druecke, Oshkosh, WI (US); Blake A. Hondl, Dunwoody, GA (US); Jonathan Kyle Arendt, Hortonville, WI (US)

Correspondence Address: KIMBERLY-CLARK WORLDWIDE, INC.;Tara Pohlkotte, 401 NORTH LAKE STREET, NEENAH, WI, 54956, US

(21) Appl. No.: 12/215,442

(22) Filed: Jun. 26, 2008

(51) Int. Cl. A61F 7/00

(52) U.S. Cl. 607/111

(57) ABSTRACT

A therapeutic delivery system that includes an appliance for selectively holding at least one therapeutic article in contact with at least a portion of a user's body. The therapeutic article may contain an exothermic compound that releases heat after selective activation by the user. The therapeutic article may be selectively located on the appliance by the user. The rate at which the exothermic composition reacts with a catalyst may be selectively controlled by the user. The appliance may be a garment that covers or encloses a portion of the user's body.

### BACKGROUND

The present invention relates to a disposable appliance for removably holding at least one therapeutic article (heating or cooling) in close bodily contact. More particularly, the invention relates to a disposable garment constructed to enclose or cover a portion of the body, and a therapeutic article with which the user may selectively position on the garment.

A common method of treating acute, recurrent, and chronic pain is by the topical application of heat to the afflicted area. Such heat treatments are used as therapy for aches, stiffness in muscles and joints, nerve pain, rheumatism and the like. The method for relieving pain often involves topically applying a relatively high heat, e.g., greater than about 40 degree C., for a short period of time, such as from about twenty minutes to about one hour.

Combinations of elastic wraps and heating pads are available for treating pain. Many of these combination devices use hot water bottles, hot packs, and the like, and are reusable by heating the contents, such as water and/or microwaveable gels. Many such heating devices require the thermal source to be replenished and are inconvenient to use on a regular or extended basis. Further, the heat energy may not be immediately available when needed or released in a controllable manner. Such thermal devices may not provide long-lasting heat or maintain a consistent temperature over long periods of time, and tend to only grow cooler over time.

Disposable heat packs based on iron oxidation, such as those described in U.S. Pat. Nos. 4,366,804, 4,649,895, and 5,046,479, are known. However, many of these devices are bulky, have difficulty staying in place during use, and/or have unsatisfactory physical dimensions that hinder their effectiveness. Such devices cannot be easily incorporated into wrap- or tube-holders that comfortably conform to various body contours.

Thus, there is a continuing need for a convenient and customizable appliance for holding therapeutic articles in close contact with the body.

### SUMMARY

In one aspect, the present invention is a therapeutic delivery system for the body of a user, the system including a single-use disposable appliance for selectively holding at least one therapeutic article against the user's body. The appliance is constructed to enclose or cover at least a portion of the body in need of treatment. At least one therapeutic article can be selectively positioned by the user on the appliance. The appliance has a body-facing side, an exposed side opposite the body-facing side, and a therapeutic agent on the body-facing side so that the appliance can deliver an effective amount of the therapeutic agent to the portion of the body in need of treatment.

In another aspect of the present invention there is a therapeutic delivery system for a user's body that includes a single-use disposable appliance for selectively holding a therapeutic article containing an exothermic composition directly or indirectly against the user's body. The appliance is a garment constructed to cover or enclose at least a portion of the user's body. The therapeutic article comprises a body-facing side and an outer side, with a first selective-activation member that removably covers at least a portion of the outer side, and a second selective-activation member that removably covers at least a portion of the first selective-activation member.

### DRAWINGS

FIG. 1 is a cut-away plan view of a first embodiment of the system of the present invention.

FIG. 2A is a plan view of a second embodiment of the system of the present invention, wherein therapeutic articles are placed in a first position.

FIG. 2B is a plan view of the embodiment of FIG. 2A, wherein therapeutic articles are placed in a second position.

FIG. 3 is a side view of a third embodiment of the system of the present invention.

FIG. 4 is a side view of a fourth embodiment of the system of the present invention

FIG. 5 is a side view of a material substrate that may be used to make any of the embodiments of the present invention.

FIG. 6 is a cross-sectional view of a first embodiment of the therapeutic article shown in FIG. 3, taken at lines 6-6.

FIG. 7 is a cross-sectional view of a second embodiment of the therapeutic article shown in FIG. 3, taken at lines 7-7.

FIG. 8 is a plan view of a fifth embodiment of the system of the present invention.

FIG. 9 is a cross-sectional view of one embodiment of the therapeutic article, showing a selective activation member thereon.

FIGS. 10(a-c) are plan views of alternative embodiments of the pouch shown in FIG. 8.

FIG. 11 is a plan view of another embodiment of a therapeutic article made with an array of heat cells.

FIG. 12 is a side view of the therapeutic article of FIG. 11.

FIG. 13 is a plan view of yet another embodiment of a therapeutic article made with an array of heat cells with selective activation members.

### DESCRIPTION

### Overview

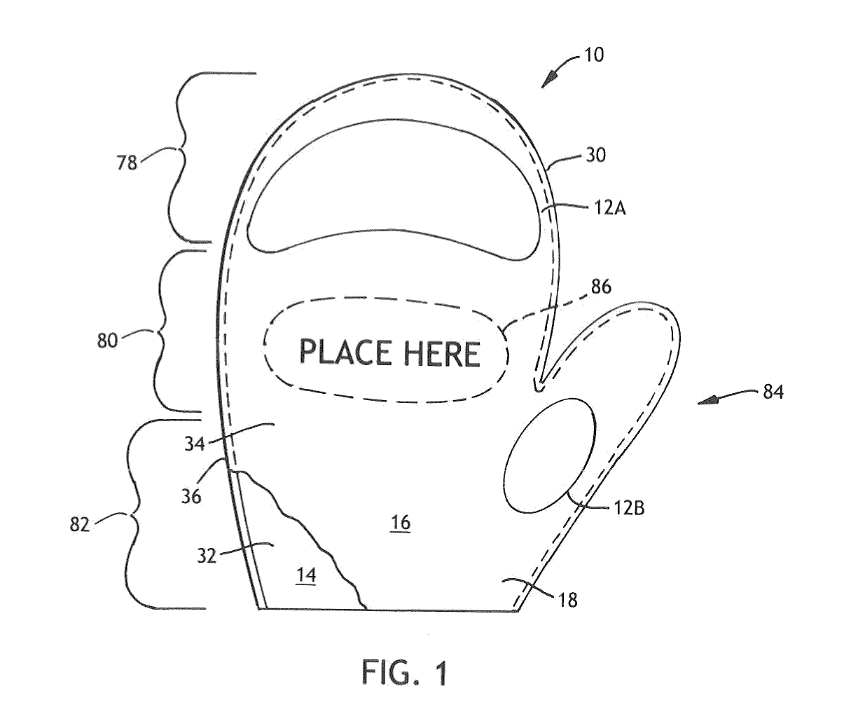

The present invention shown in FIGS. 1-13 is a therapeutic delivery system 10 that includes a single-use disposable appliance 30 for selectively holding at least one therapeutic article 12 in bodily contact with a user. (The term “disposable” is defined as an item that is used a single time by a single user before being disposed.)

A user may selectively position the therapeutic article 12 on a preferred area of the body. The appliance 30 includes a body-facing side 14 and an exposed side 16 opposite the body-facing side 14. The appliance 30 may include a therapeutic agent at the body-facing side 14 so that the appliance 30 is capable of delivering an effective amount of the therapeutic agent to the portion of the body in need of treatment.

The appliances 30 herein are coordinated to have compatible shapes, sizes, and flexibility in order to fit reliably and comfortably against the body region where thermal, therapeutic or other benefit is desired. The appliance 30 can be used with a heating article to provide heat to the body. The appliance 30 can also be used to deliver a therapeutic agent, such as an aromatic compound, a pharmaceutical active, a lotion, an emollient, a moisturizing agent, or mixtures thereof, to the body.

The therapeutic article 12 herein can be used in conjunction with an appliance 30 for relieving pain in various places on the body, including lower back pain; hand or foot pain; arm or leg pain, e.g., in the thigh, knee, forearm; and neck and shoulder pain. In another embodiment, the therapeutic article 12 can be used for improving thermal comfort by heating various body regions, including the core of the body, head, etc.

The system of the present invention may allow the therapeutic article 12 to (a) achieve a desired level of efficacy (e.g., heat) for a period of time, and/or (b) have extended or repeated exposure time as desired.

### Representative Appliance Configurations

The appliance 30 is generally a garment constructed to cover or enclose a body portion of the user where treatment is desired. In one embodiment of the present invention, the appliance 30 is constructed to fit an appendage such as a hand or foot, and thus, may be in glove/mitten or sock form, respectfully, see FIGS. 1-3. In yet another embodiment (not shown), the appliance 30 may be constructed into a wrap or patch that can be selectively fastened about a user's limb, torso, or head. Specifically, one or more substrates, such as those described above, may be configured into the form of a glove, mitten, sock, sleeve, patch, or other article designed to be fitted to a part of the body. Examples of suitable appliances 30 and substrates may be seen in U.S. patent application Ser. No. 11/190,597, APPLIANCE FOR DELIVERING A COMPOSITION, filed Jul. 26, 2005, incorporated herein to the extent that it is consistent with the present invention.

FIG. 1 representatively depicts a mitten-shaped appliance 30 comprising a first piece (or substrate) 32 attached to a second piece (or substrate) 34 at a location proximate to the perimeters of these two substrates. In this representative illustration, the two substrates 32, 34 are attached to one another mechanically by sewing the pieces together at a location proximate to the perimeters of the two substrates. The resulting appliance was then inverted so that a seam 36 formed by sewing the substrates together is on the interior of the appliance. Of course the finished appliance need not be inverted; the seam 36 can remain on the exterior of the appliance. Note, too, that the individual pieces need not be joined in a way that produces a seam 36. The edges of the individual pieces may be butted together, and then, for example, joined and/or welded together using ultrasonic energy, heat, or solvents. Alternatively, the individual pieces may be butted together, and another material, such as an adhesive or an adhesive tape, used to join the pieces together.

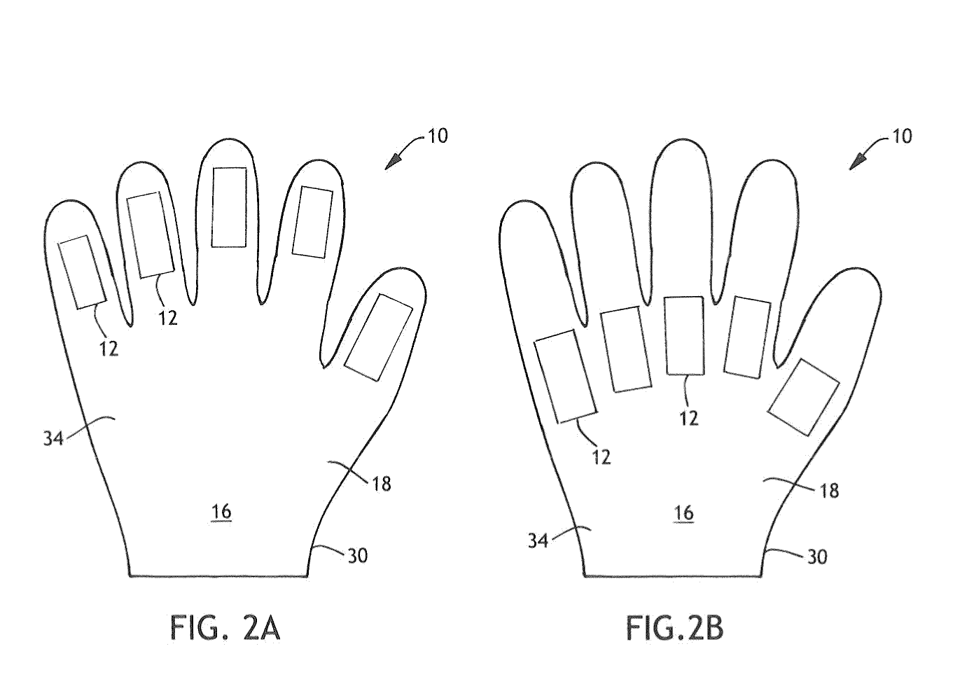

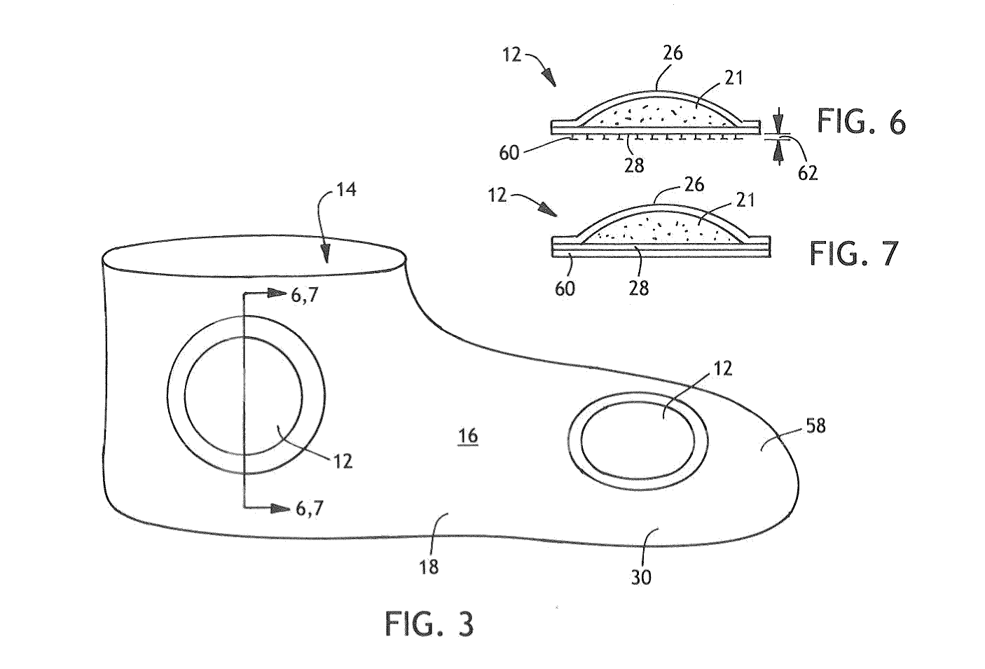

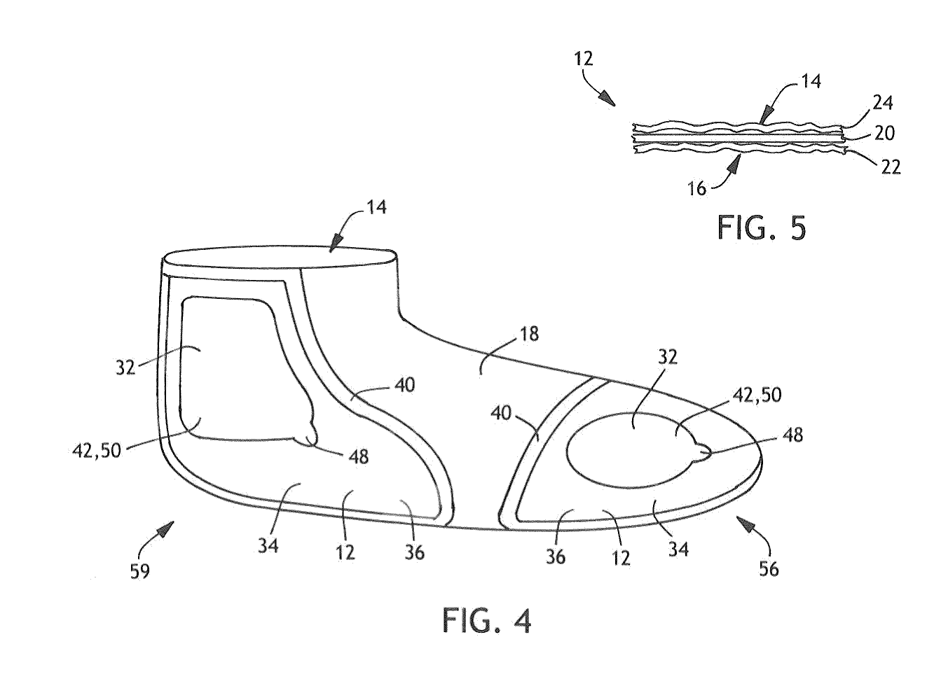

FIGS. 2A and 2B representatively depict an appliance 30 in a glove shape, and FIGS. 3 and 4 depict the appliance 30 in a sock shape. However, it is noted that individual pieces (or substrates) may be cut into a variety of shapes and sizes. The pieces may be cut so that the resulting appliance is in the shape of a tube, sleeve, patch, or the like. Any shape is possible, and the resulting appliance may or may not define an interior volume into which a user may insert a portion of his or her body (e.g., a finger, toe, hand, foot, wrist, forearm, etc.) such that a composition applied to the bodyfacing side 14 of the appliance 30 may be transferred to skin or tissue in contact therewith.

The individual substrates or pieces making up the appliance 30 need not be sewn together. The individual pieces or substrates may also be joined ultrasonically, thermally, adhesively, cohesively, using tape, by fusing the materials together (e.g., by using an appropriate solvent), by welding the materials together, or by other approaches. So long as the individual pieces or substrates remain attached or connected during normal use of the appliance 30, and attachment or connection is such that the composition or formulation on the interior surface of the appliance is contained within the appliance 30 (i.e., there is minimal or no leakage of the formulation or composition), any connection or attachment method may be used.

Alternatively, a substrate could be prepared in the form of a rectangle, oval or other shape (not shown). An adhesive capable of adhering to skin could then be applied to all or part of the perimeter of the shape such that the appliance 30 could be releasably adhered to the skin. The composition to be transferred to the skin could then be coated or deposited on the bodyfacing surface 14 of the appliance 30.

In the alternative, an appliance 30 may be formed from a single piece of substrate, and formed by folding the substrate back on itself, see U.S. patent application Ser. No. 11/190,597, previously incorporated. As with two (or more) pieces that may be joined together to form an appliance 30 of the present invention, a single piece may be joined to itself using any of the approaches discussed above.

### Representative Substrates for Constructing an Appliance of the Present Invention

A substrate used to make an appliance of the present invention will generally have three layers: a water-impermeable layer, such as a film, sandwiched between two fibrous layers, such as nonwoven materials. An example of such a substrate 18 is depicted in FIG. 5, which representatively illustrates a water-impermeable layer 20 attached to an outer fibrous layer 22 and an inner fibrous layer 24. The material for the outer fibrous layer 22 may be any material that provides for a cloth-like appearance (as opposed to, for example, a smooth or rubbery appearance as in a neoprene rubber glove). The material for the inner fibrous layer 24 may be any material that is fibrous in nature, such as a nonwoven material. The inner fibrous layer may possess an uneven, undulating surface to help contain the formulation or composition applied to the surface of the inner fibrous layer 24. As noted above, the rugosity of this inner material can be achieved or enhanced by attaching the inner fibrous layer 24 to the water-impermeable layer 20 at discrete points or locations (e.g., by thermally point bonding the materials together, as is discussed in more detail below) while the water-impermeable layer 20 is in a stretched condition. When the water-impermeable layer 20 (and, therefore, the resulting laminate) is allowed to relax, the inner fibrous layer 24 is gathered to produce undulations in the inner fibrous layer. Of course, both the inner fibrous layer 24 and the outer fibrous layer 22 are gathered in this way if they are attached to the water-impermeable layer 20 at discrete points or locations while the water-impermeable layer 20 is in a stretched condition (and then allowed to relax).

The inner and outer fibrous layers may be the same or may be different. Generally the water-impermeable layer 20 is elastomeric, with the resulting substrate 18 able to stretch and conform to a hand, foot, extremity, or other body region to which the appliance is applied.

If a nonwoven material is used to make the inner and outer fibrous layers, then commercially available thermoplastic polymeric materials can be advantageously employed in making the fibers or filaments from which the outer fibrous layer 22 and inner fibrous layer 24 are formed. As used herein, the term “polymer” shall include, but is not limited to, homopolymer, copolymers, such as, for example, block, graft, random and alternating copolymers, terpolymers, etc., and blends and modifications thereof.

Nonwoven webs that can be employed as the nonwoven layers 22 and 24 of the present invention can be formed by a variety of known forming processes, including spunbonding, airlaying, meltblowing, or bonded carded web formation processes. Spunbond nonwoven webs are made from melt-spun filaments. The production of spunbond nonwoven webs is described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,538 to Peterson, and U.S. Pat. No. 3,542,615 to Dobo et al., all of which are incorporated herein by reference.

The melt-spun filaments formed by the spunbond process are generally continuous and have average diameters larger than 7 microns based upon at least 5 measurements, and more particularly, between about 10 and 100 microns. Another frequently used expression of fiber or filament diameter is denier, which is defined as grams per 9000 meters of a fiber or filament.

The water-impermeable layer 20 can be formed of any film that can be suitably bonded or attached to top and bottom layers 22 and 24 respectively to yield a substrate 18 having the performance characteristics and features described herein. A suitable class of film materials includes a thermoplastic elastomeric polyolefin polymer. These (and other) components can be mixed together, heated and then extruded into a mono-layer or multi-layer film using any one of a variety of film-producing processes known to those of ordinary skill in the film processing art. Such film-making processes include, for example, cast embossed, chill and flat cast, and blown film processes.

Typically the water-impermeable layer 20 will be attached to the outer fibrous layer 22 and inner fibrous layer 24 by thermally bonding the three layers together at discrete points (see, e.g., discussion in preceding paragraph as well as U.S. Pat. No. 6,037,281, entitled “Cloth-Like, Liquid-Impervious, Breathable Composite Barrier Fabric,” to Mathis, et al.). As noted above, the two fibrous layers may be bonded or attached to the water-impermeable layer at discrete locations while the water-impermeable layer is in a stretched condition, thereby producing undulations when the resulting laminate is in a relaxed condition. Other known means for bonding and laminating the water-impermeable layer 20 to fibrous layers 22, 24 may be used, provided the resulting substrate 18 has the required properties described herein. For example, the three layers may be adhesively bonded to one another.

In addition to the polyolefin polymer, the substrate 18 can also include one or more fillers. As used herein, a “filler” is meant to include particulates and other forms of materials which can be added to the film polymer extrusion blend and which will not chemically interfere with the extruded film, but which are able to be uniformly dispersed throughout the film. Suitable fillers are described in U.S. patent application Ser. No. 11/190,597, previously incorporated herein.

The film layer used in the example of the present invention described below is a mono-layer film, however, other types, such as multi-layer films, are also considered to be within the scope of the present invention provided the forming technique is compatible with films described herein.

The preceding paragraphs describe some examples of a substrate that may be used in an appliance of the present invention. For additional examples, see, e.g., U.S. Pat. No. 6,037,281, entitled “Cloth-Like, Liquid-Impervious, Breathable Composite Barrier Fabric,” to Mathis, et al.; U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference in a manner consistent herewith. Any 3-layer substrate may be used, but it is desirable that the outer fibrous layer presents a cloth-like appearance and feel; the inner fibrous layer is sufficiently rugose to help contain the formulation or composition applied to the interior of the appliance (to minimize leakage of the formulation or composition from the appliance); and the water-impermeable layer—such as a film—is capable of minimizing evaporation or transfer of water through the appliance.

### Therapeutic Agents

The therapeutic agent is transferable to the wearer's body in an effective amount to provide a desired therapeutic benefit, and may include an aromatic compound, a pharmaceutical active, a lotion, an emollient, a moisturizing agent, or mixtures thereof. Other formulations or compositions that may be used with an appliance of the present invention include emulsifiers, surfactants, viscosity modifiers, natural moisturizing factors, antimicrobial actives, pH modifiers, enzyme inhibitors/inactivators, suspending agents, pigments, dyes, colorants, buffers, perfumes, antibacterial actives, antifungal actives, pharmaceutical actives, film formers, deodorants, opacifiers, astringents, solvents, organic acids, preservatives, drugs, vitamins, aloe vera, and the like.

In some versions of the invention, a clinically beneficial additive of the formulation or composition may either interact directly with epithelial tissue at the cellular level to provide a benefit to the skin, or alternatively, may interact with components at or near the skin surface in order to provide a benefit to the skin.

In one embodiment, the clinically beneficial additive may be an emollient, which is herein defined as an agent that helps restore dry skin to a more normal moisture balance. Emollients that may be suitable for use with the present invention include beeswax, butyl stearate, cermides, cetyl palmitate, eucerit, isohexadecane, isopropyl palmitate, isopropyl myristate, mink oil, mineral oil, nut oil, oleyl alcohol, petroleum jelly or petrolatum, glyceral stearate, avocado oil, jojoba oil, lanolin (or woolwax), lanolin derivatives such as lanolin alcohol, retinyl palmitate (a vitamin A derivative), cetearyl alcohol, squalane, squalene, stearic acid, stearyl alcohol, myristal myristate, certain hydrogel emollients, various lipids, decyl oleate and castor oil.

Humectants that may be suitable for use with the present invention include alanine, glycerin, PEG, propylene glycol, butylenes glycol, glycerin (glycol), hyaluronic acid, Natural Moisturizing Factor (a mixture of amino acids and salts that are among the skin's natural humectants), saccharide isomerate, sodium lactate, sorbitol, urea, and sodium PCA.

Other clinically beneficial agents that may be suitable for use with the present invention include antioxidants, a unique group of substances that protect a body or other objects from oxidizing. Antioxidants prevent or slow the oxidation process, thereby protecting the skin from premature aging. Exemplary antioxidants for use in the present invention include ascorbic acid ester, vitamin C (ascorbic acid), vitamin E (lecithin), Alpha-Glycosyl Rutin (AGR, or Alpha Flavon, a plant-derived antioxidant), and coenzyme Q10 (also known as ubiquinone).

Other clinically beneficial agents which may be delivered to the skin during use include chelating agents, such as EDTA; absorptive/neutralizing agents, such as kaolin, hectorite, smectite, or bentonite; other vitamins and vitamin sources and derivatives, such as panthenol, retinyl palmitate, tocopherol, and tocopherol acetate; and anti-irritants such as chitin and chitosan.

Additional examples of beneficial agents include skin conditioners, which are herein defined as agents that may help the skin retain moisture, improve softness, or improve texture. Skin conditioners include, for example, amino acids, including alanine, serine, and glycine; allantoin, keratin, and methyl glucose dioleate; alpha-hydroxy acids, including lactic acid and glycolic acid, which act by loosening dead skin cells from the skin's surface; moisturizers (agents that add or hold water in dry skin), including echinacea (an extract of the coneflower plant), shea butter, and certain silicones, including cyclomethicon, dimethicone, and simethicone.

Other examples of beneficial botanical agents, extracts, or other materials that may be suitable for use with the present invention include almonds, chamomile extracts such as bisabolol (believed to relieve irritation, swelling and itching in the skin), elder flowers, honey, safflower oil, and elastin (safflower oil and elastin are believed to aid in retaining skin elasticity).

In addition to one or more clinically beneficial additives, other additives may be included in the formulation or composition. For example, a silicone polymer may be included to improve the slip characteristics of the elastomeric article. Possible silicone polymers include reactive silicones, non-reactive silicones, or a mixture of reactive and non-reactive silicones. Suitable silicones may include, for example, aminosilicones, polyether-modified amino silicones, amino-substituted siloxanes having terminal hydroxy groups, epoxy silicones, quaternary silicones, dimethicone, silicone polyethers, polyether epoxy silicones, silanol fluids, polysiloxy linoleyl pyrrolidone phospholipids, and combinations of possible silicones.

Other additives may be included, for example, glucose derived polymers, or mixtures containing glucose derived polymers (e.g., lauryl glucoside available from Cospha under the trade designation Planteran PS 400), silica, silica dispersions, wetting agents, and preservatives (i.e., parabens, such as methylparaben and propylparaben). In one embodiment, the personal-care composition may include emulsion stabilizers. Exemplary emulsion stabilizers include aluminum stearate, magnesium sulfate, hydrated silica, and ozokerite.

In another embodiment, a beneficial agent may be held in the formulation or composition in liposomes. A liposome is a vehicle for delivering agents to the skin. More specifically, a liposome is a microscopic sphere formed from a fatty compound, a lipid, surrounding a water-based agent, such as a moisturizer or an emollient. When the liposome is rubbed into the skin, it releases the agent throughout the stratum corneum.

In another embodiment, the beneficial agent may be present in the carrier in the form of a microencapsulant. A microencapsulant is a sphere of an emollient surrounded by a gelatin membrane that prevents the emollient from reacting with other ingredients in the coating composition and helps distribute the emollient more evenly when pressure is applied and the membrane is broken.

The process of forming these beads is called microencapsulation and is generally known in the art.

The formulation or composition of the present invention may be applied to the appliance as an aqueous solution, a dispersion, or an emulsion. In one embodiment, an aqueous composition may be formed including from about 4.5% to about 6% by weight of a humectant. In other embodiments, the humectant may be present at 30% or more by weight. In some other embodiments, the humectant may be present at about 10% to about 20% by weight. In still other embodiments, the humectant is present at about 5% to about 40% by weight. This composition may then be applied to the interior surface of an appliance of the present invention.

In one embodiment, the personal-care composition may be applied as an emulsion. In one embodiment, the formulation or composition may be applied to the surface of the appliance as a micro-emulsion. A micro-emulsion is a particularly fine-particle emulsion that can be applied in a spray form. The particle size of a micro-emulsion is generally less than about one micron, whereas traditional emulsions demonstrate particle sizes of greater than about 50 microns.

The components of a formulation or composition may be applied in combination or separately to the surface of the appliance. For example, a 100% humectant composition may be applied, followed by another 100% beneficial additive composition, such that the two (or more) separate applications together form the coating of the appliance. In such a manner, layers of additives may be built up on the surface of the appliance.

### Therapeutic Articles

As used herein, the term “therapeutic article” is an article placed against or in proximity to the body in a specific region to transfer heat to the user's body for pain relief or thermal comfort. See, U.S. Pat. No. 6,146,732, Davis, et al.; U.S. Pat. No. 6,074,413, Davis, et al.; U.S. Pat. No. 6,336,935, Davis et al; and U.S. Pat. No. 6,020,040, Cramer et al.; all incorporated herein by reference to the extent they are consistent with the present invention. The therapeutic articles 12 typically comprise one or more heat cells. The term “heat cell” refers to a unified structure comprising an exothermic composition 21, typically having specific iron oxidation chemistry, enclosed within at least two layers. However, it is contemplated that other exothermic compositions may be used that may or may not use oxygen as a catalyst. As oxygen will be the most common catalyst, it is referred to specifically, though it is intended that oxygen could be substituted depending on the chemistry 21. Referring to FIG. 6, at least one exposed layer 26 may be oxygen permeable, capable of providing long lasting heat generation with temperature control, and have specific physical dimensions and fill characteristics, such as described in U.S. Pat. No. 5,918,590, Burkett, et al., incorporated herein by reference to the extent they are consistent with the present invention. A backing layer 28 may be made from the same material as the exposed layer 26, or may be an air impermeable layer so that the oxidation may only occur as oxygen enters the exposed layer 26.

As used herein, the term “air impermeable” with respect to the therapeutic article 12 of the present invention refers to a material wherein oxygen or other catalytic gases pass through at such an insignificant rate that it does not cause the composition 21 contained within the therapeutic article 12 to activate prematurely or become spent prior to a predetermined shelf life. This allows the therapeutic article 12 to be stored for several months or years prior to use.

As used herein, the term “air permeable” refers to a material that is not considered to be air impermeable.

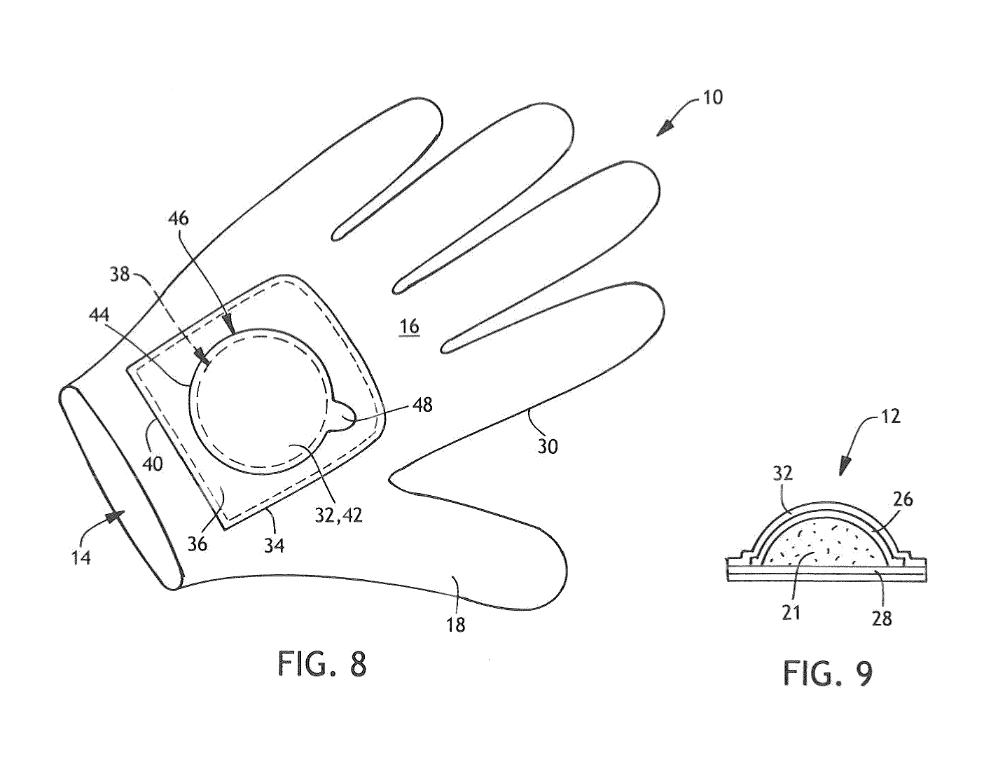

Referring to FIG. 8, the therapeutic article 12 (not seen) may include a selective activation member 32. A selective activation member 32 is a removable member (such as a film) that is placed over any therapeutic article layer that is air permeable, such as exposed layer 26, or possibly, the backing layer 28. The selective activation member 32 allows a user to control the rate at which the exothermic reaction in the therapeutic article will occur.

Selective activation member 32 may be air impermeable or air permeable, depending on whether it is used (a) to control the activation of composition 21, or (b) to control the rate of activation of composition 21. “Activation” results when oxygen or other catalysts are allowed to initiate the exothermic reaction. “Rate of activation” can be thought of as a number of the units of oxygen or other catalysts that reach the composition 21 in a given period of time. For example, when a unit of catalyst makes contact with the composition 21 contained between exposed layer 26 and backing layer 28, the therapeutic article will produce heat at a first rate. If two units of catalyst make contact with the composition 21, the therapeutic article will produce heat at a second rate that is faster than the first rate.

It is contemplated that the two different types of selective activation numbers 32 may be combined with or may replace the components of the therapeutic article 12. For example, an activation-control number, referred to as a barrier 42 may cover one or more activation-rate-control members, referred to as semi-barrier 50. In addition, a barrier 42 may instead be part of an air-tight package used to contain a therapeutic article 12. Many combinations are possible and several non-limiting examples are shown and discussed below.

In one embodiment of the present invention, as shown in FIG. 8, a pouch 34 is used to contain a therapeutic article 12 and maintain its position against the exposed side 16 of appliance 30. The pouch, 34 is made with a sheet member 36 that is an air impermeable material. A sheet member 36 may be sealed to another air impermeable material, such as the appliance substrate 18, to create an air-tight package in which a therapeutic article 12 is contained.

Sheet member 36 may be sealed to the substrate 18 using ultrasonic, pressure, or thermal methods. In the alternative, an adhesive may be used. In any of these methods, an air impermeable bond should result so that the therapeutic article 12 remains inactivated until purposefully activated by a user. A margin 40 occurs about the perimeter of the pouch 34 where it is sealed to the substrate 18 or other piece, described above.

Suitable materials for sheet member 36 include polyester, polyethelene, polyvinyl chloride or other plastic films that are substantially air impermeable, at least for a predetermined shelf life.

Still referring to FIG. 8, sheet member 36 may include an opening such as a window defined by an edge 38. The opening serves to allow air to reach the therapeutic article 12. The opening may be of any shape desired and may be strictly functional or possibly aesthetic. For instance, the window may be an angular shape instead of a round shape and may have a decorative shape such as a flower or star. Regardless, the configuration of edge 38 is such that the therapeutic article 12 cannot accidentally fall out of pouch 34 through the window.

So that the composition 21 is not activated until a user is ready, the window is sealed by a barrier 42. Barrier 42 may be a flexible sheet that is separate or integral to the sheet member 36. If it is a separate item, the barrier 42 has an area that is larger than the window to create an overlap margin 44 between edge 38 and barrier edge 46. The barrier edge 46 may define a shape similar to that defined by edge 38, or in the alternative, may be shaped to include features such as a pull tab 48. Pull tab 48 may not be adhered to the sheet member 36 so that it may be grasped to peel away the barrier 42 from the sheet member 36. It is further contemplated that the barrier 42 may have an aesthetic shape, including but not limited to a flower, octagon, starburst, or the like. Barrier 42 is connected to the sheet member 36 at the overlap margin 44 using an adhesive or a thermal bond. An example of a suitable adhesive may be a hot melt adhesive or the like.

In an embodiment where barrier 42 is integral to sheet member 36 (not shown), the edge 38 will have the same shape as edge 46, and only be separated by a line where a stress concentration occurs. In this case, the barrier 42 and sheet member 36 are made from the same sheet member 36. The stress concentration is created on sheet member 36 by mechanical, hydraulic, chemical, or ultrasonic methods. For instance, the sheet member may be scored about edge 38 to create weakness. When the score is broken, the barrier 42 may be removed by tearing it away from the sheet member 36 at the score line.

In operation, a user removes the barrier 42 from the window so that the composition 21 may be activated. It is contemplated that in the embodiments shown in FIGS. 4 and 8, the system 10 could be stored in a separate airtight package until ready for use, and the selective activation member 32 could instead be a semi-barrier 50. In addition, in this embodiment, the sheet member 36 could be air permeable.

Referring now to FIG. 4, depicted is an example of an appliance 30 in the form of a sock. Appliance 30 has regions where specially shaped pouches 34 are situated at a toe region 56 and a heel region 59. This depiction exemplifies that the pouches 34, barriers 42, and underlying windows (not shown) can be shaped to fit specific anatomy. It is noted that the barriers 42 may instead be semi-barrier(s) 50, as described below.

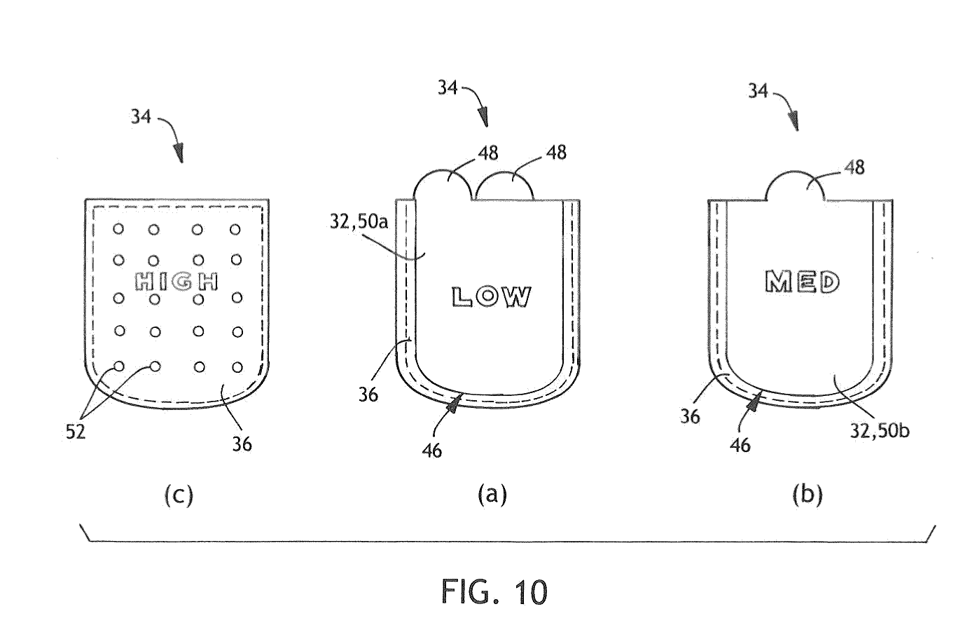

In another embodiment of the present invention, the pouch 34 has multiple removable layers so that a user may select whether or not the composition 21 will be exposed to air relatively slowly or quickly. Referring now to FIGS. 10(a-c), depicted is a pouch 34 similar to that shown in FIG. 8. In this embodiment, however, the pouch 34 is used in conjunction with one or more semi-barriers 50, which are air permeable. Without the semi-barriers 50 covering the sheet member 36, the rate at which air may contact the contained composition 21 is at its maximum. When one or more layers of semi-barrier 50 used to cover a window, or in this case, an array of apertures 52, the rate at which air may contact the composition 21 is retarded.

For example, depicted in FIG. 10(a) is a pouch 34 that has two layers of semi-barrier 50 that cover the pouch apertures 52. For ease of discussion, the outermost semi-barrier is reference 50a, and the underlying semi-barrier is reference 50b. In relative terms, when semi-barriers 50a and 50b are covering the apertures 52, the composition 21 is making contact with air at a low rate. Referring now to FIG. 10(b), the pouch 34 has only one layer of semi-barrier 50, namely sem-barrier 50b. Again, in relative terms, when only semi-barrier 50b is covering apertures 52, the composition 21 is making contact with air at a medium rate. Referring now to FIG. 10(c), there are no semi-barriers 50 present, and the composition 21 is making contact with air at a relatively high rate. Indicia 54 my be placed on the semi-barriers 50a and 50b and sheet member 36 to indicate to a user the relative rate at which air is contacting composition 21, which is directly related to the speed at which the composition will heat. Indicia 54 may be alpha-numeric characters, color coding, a texture such as Braille, or a combination thereof.

Semi-barriers 50a and 50b each have a layer of adhesive in the shape of a frame surrounding the perimeter of the barriers. The adhesive will allow the semi-barriers 50a and 50b to stick to each other but not occlude the passage of air through the semi-barriers 50a and 50b. It is contemplated that an air permeable adhesive could cover the entire surface of each semi-barrier 50a and 50b so that they can more uniformly adhere to other semi-barriers.

It is contemplated that the apertures 52 shown in FIG. 10(c) could be replaced by an air-permeable sheet. Further, it is contemplated that the air-permeable semi-barriers 50 could be replaced by air impermeable sheets having apertures 52 therein. If the semi-barrier 50 and the sheet member 36 each had apertures 52, the apertures 52 in the layers would need to be aligned for air to transfer to the composition 21. Of course, there would be fewer apertures 52 in semi-barrier 50b than in sheet member 36, and fewer apertures 52 in semi-barrier 50a than in semi-barrier 50b.

Each of the semi-barriers 50 may have tabs 48 extending therefrom. The tabs 48 may have indicia thereon, and may be staggered as shown. However, tabs 48 may extend from any edge 46 and may be stacked or aligned with one another if desired.

Suitable materials for the semi-barriers 50 include nonwoven, spunbond, apertured films (e.g., polyester, polyethylene, or PVC films), or the like. Because semi-barriers 50 cannot prevent the composition 21 from reacting with air, an additional barrier 42 needs to be placed over the outermost semi-barrier 50, similar to that described previously, or the entire appliance 30 needs to be contained within an air-tight container until ready for use.

Referring to FIGS. 3, 6, and 7, in another embodiment of the present invention, at least one therapeutic article 12 is capable of being selectively positioned on the appliance 30 with the use of a mechanical fastener 62, such as hook, microhook, reticulated foam, or the like, or with the use of an adhesive layer 64. In this case, the appliance 30 has a first fastening material 58 and the therapeutic article 12 comprises a second fastening material 60 that cooperatively engages the first fastening material 58 to enable the therapeutic article 12 to be removably affixed to exposed side 16 of appliance 30. This allows the user to place the therapeutic article to a position of choice. It may also allow the user to move the therapeutic article 12 from an initial position during use. It is noted that the first fastening material may be substrate 18, if substrate 18 has an inherent characteristic that allows it to function as a fastening material. For example, it may have a loop structure that will connect with the substrate 18 hook member.

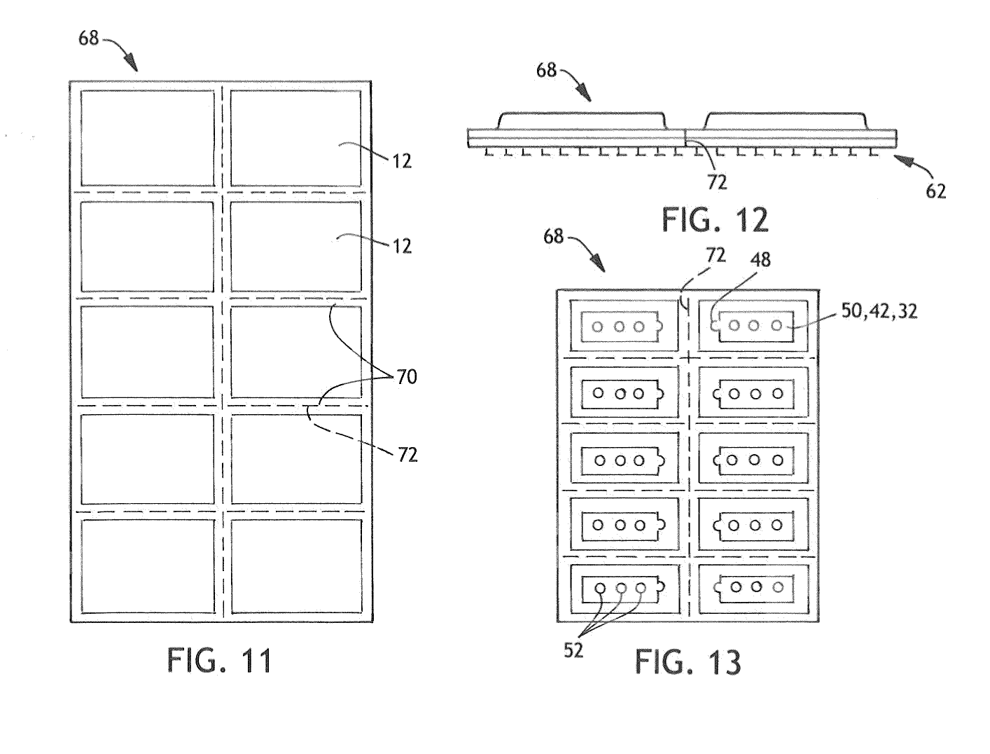

In another embodiment of the present invention, a therapeutic article 12 is attached to one or more therapeutic articles 12, desirably by a frangible bond to create an array. Referring now to FIG. 11, an array of therapeutic articles is formed so that a margin 70 separates each therapeutic article 12. A line 72 of perforations is the frangible bond. By having each therapeutic article 12 operate as a separate heat cell, a user may create custom-sized/shaped panels of therapeutic articles. A user may decide to leave each panel connected as shown in FIG. 11, or go so far as to separate each one and place them on an appliance. For example, each of the therapeutic articles 12 have been separated from one another and placed on the glove-style appliance 30 shown in FIGS. 2A and 2B. FIG. 12 is a side view of the array shown in FIG. 11. In this example, a hook-style mechanical fastener 62 is shown. However, as mentioned previously, other types of mechanical fasteners or adhesive may be used.

Referring now to FIG. 13, the array shown in FIG. 11 may have barriers 42 on each individual therapeutic article 12, and/or one or more semi-barrier 50. A user may desire that each cell heat at a different rate, as described above.

FIGS. 2A and 2B demonstrate only one way in which the customizable panels 68 may be utilized. The therapeutic articles may be placed on other articles, or on any relatively flat portion of the appliance 30. It can be seen that on the glove of FIG. 2A, a user chose to place each of the therapeutic articles 12 over the two knuckles closest to the finger tips. On the glove of FIG. 2B, the user chose to place each therapeutic article 12 over the joints farthest away from the finger tips.

In one embodiment, the appliance 30 may have instructions or indicia that suggest a particular location of a therapeutic article 12. For example, referring to FIG. 1, a mitten-style appliance 30 is shown with a therapeutic article 12A located over an upper region 78 of the appliance 30. Therapeutic article 12A could instead be located in the middle region 80, or the lower region 82. The user could also orient a therapeutic article 12 in the thumb region 84. Desirably, the therapeutic article 12A may be located anywhere on the exterior surface 16 of the appliance 30, provided that the surface is large enough to accommodate a therapeutic article.

Furthermore, one or more additional therapeutic articles, such as therapeutic article 12B, could be located on the appliance 30. The additional therapeutic articles could be identical or different in shape and/or size.

The exposed side 16 of appliance 30 may also include placement indicia. For example, as seen in FIG. 1, placement indicia 86 may direct the user to a preferred placement for an article 12. The indicia may coincide with portions of the body known to benefit from heat therapy. For instance, a person suffering from certain forms of arthritis may prefer therapeutic article 12 be placed over the knuckles closest to the palm of hand. Placement indicia 86 may consist of alphanumeric characters, design colors, textures, or a combination thereof. Indicia 86 may provide information as to particular ailments that might benefit from having a therapeutic article 12 placed over that particular indicia. In addition to, or in lieu of placement indicia 86, a set of written instructions may be included with a therapeutic delivery system 10 of the present invention.

While particular embodiments of the present invention have been illustrated and described, various other changes and modifications can be made without departing from the spirit and scope of the invention. It is therefore intended to cover all such changes and modifications that are within the scope of this invention.

1. A therapeutic delivery system for the body of a user comprising:
a single-use disposable appliance for selectively holding at least one therapeutic article against the user's body, said appliance constructed to enclose or cover at least a portion of the body in need of treatment; and
wherein the at least one therapeutic article can be selectively positioned by the user on said appliance,
wherein the appliance comprises a body-facing side, an exposed side opposite the body-facing side, and a therapeutic agent on the body-facing side; wherein the appliance can deliver an effective amount of the therapeutic agent to the portion of the body in need of treatment.

2. A system according to claim 1 wherein the therapeutic article comprises an exothermic composition.

3. A system according to claim 1 wherein the appliance comprises at least one pocket for containing the therapeutic article, the pocket having a window therein to allow air to contact the exothermic composition.

4. A system according to claim 1 wherein the therapeutic article comprises a fastening member that cooperatively engages the appliance and enables the therapeutic article to be selectively and removably affixed to the exposed side of the appliance.

5. A system according to claim 1 wherein the therapeutic article is removably affixed to the appliance using an adhesive material.

6. A system according to claim 1 wherein the therapeutic agent comprises an aromatic compound, a pharmaceutical active, a lotion, an emollient, a moisturizing agent, or mixtures thereof.

7. A system according to claim 1 wherein the therapeutic article comprises a sheet with a plurality of heat cells separated from one another by a margin.

8. A system according to claim 7 wherein the margin is frangible.

9. A system according to claim 1 wherein the therapeutic article comprises a first layer of air permeable material, a second layer of air permeable or air impermeable material, and an exothermic composition located between the first layer and the second layer.

10. A system according to claim 9 wherein the therapeutic article comprises a barrier completely covering the first layer.

11. A system according to claim 10 wherein the therapeutic article comprises a selective activation member.

12. A system according to claim 11 wherein the selective activation member comprises a first semi-barrier.

13. A system according to claim 11 wherein the selective activation member comprises a second semi-barrier.

14. A system according to claims 12 comprising indicia located on the first semi-barrier.

15. A system according to claim 1 comprising placement indicia.

16. A system according to claim 1 wherein the appliance comprises a pouch, wherein the pouch comprises an exothermic composition.

17. A therapeutic delivery system for a user's body comprising:
a single-use disposable appliance for selectively holding a therapeutic article containing an exothermic composition directly or indirectly against the user's body, the appliance being a garment constructed to cover or enclose at least a portion of the user's body;
wherein the therapeutic article comprises a body-facing side and an outer side, with first selective-activation member removably covering at least a portion of the therapeutic article, and a second selective activation member removably covering at least a portion of the first selective-activation member.
